(19) United States
(12) Patent Application Publication
(10) Pub. No.: US 2024/0423776 A1
(43) Pub. Date: Dec. 26, 2024

(54) TRANSFER INSTRUMENT

(71) Applicant: TERUMO KABUSHIKI KAISHA, Tokyo (JP)

(72) Inventor: Koichi HAYAKAWA, Kanagawa (JP)

(73) Assignee: TERUMO KABUSHIKI KAISHA, Tokyo (JP)

(21) Appl. No.: 18/740,979

(22) Filed: Jun. 12, 2024

(30) Foreign Application Priority Data
Jun. 20, 2023 (JP) 2023-100702

(51) Int. Cl. A61F 2/00

(52) U.S. Cl. CPC A61F 2/0063; A61F 2002/0072

(57) ABSTRACT

A transfer instrument includes an outer cylinder and a carrier member (first carrier member) including a shaft (first shaft) and a support portion (first support portion) having a sheet shape. The support portion is retracted in the outer cylinder with the support portion deformed into a curved shape at a first position where the shaft is moved in a proximal end direction relative to the outer cylinder so as to cause the support portion to retract in the outer cylinder. When a center portion of a front surface of the first support portion in a width direction faces upward in an unfolded state of the first support portion, both sides of the front surface of the first support portion in the width direction face upward.

### CROSS-REFERENCES TO RELATED APPLICATIONS

This application is based on and claims priority to Japanese Patent Application No. 2023-100702 filed on Jun. 20, 2023, the entire content of which is incorporated herein by reference.

### TECHNOLOGICAL FIELD

The present invention generally relates to a transfer instrument for transferring a medical sheet to a treatment site of a living body.

### BACKGROUND DISCUSSION

Japanese Patent Application Publication No. 2009-000511 (JP 2009-000511 A) discloses a transfer instrument for transferring a medical sheet (cell sheet) for use in, for example, organ transplantation to a treatment site of a living body. The transfer instrument includes an outer cylinder, a slide member slidably supported in the outer cylinder, and a seat support member provided at a distal end of the slide member.

### SUMMARY

It is desired to reduce a diameter of a transfer instrument and achieve less invasion.

(1) An aspect of the disclosure includes a transfer instrument that transfers a medical sheet to a treatment site of a living body. The transfer instrument includes an outer cylinder including a distal-end opening, and a carrier member including a shaft and a support portion, with the shaft extending in an axial direction of the outer cylinder, being disposed inside the outer cylinder and being movable along the axial direction relative to the outer cylinder, with the support portion being sheet-shaped and being disposed at a distal end of the shaft. The support portion includes a support surface configured to hold the medical sheet, a front surface including the support surface, and a back surface opposite to the front surface, with the support portion having a width in a width direction orthogonal to an extending direction of the shaft, and the width of the support portion being larger than an inner circumferential length of the outer cylinder. The support portion in a first position is retracted in the outer cylinder with the support portion deformed into a curved shape. The curved shape of the support portion in the first position including a pair of separation portions spaced apart from an inner circumferential surface of the outer cylinder and protruding into the outer cylinder. The support portion is movable in a distal direction relative to the outer cylinder from the first position to a second position, in which second position the support portion protrudes distally beyond the distal-end opening of the outer cylinder and is unfolded in an unfolded state. When a center portion of the front surface of the support portion in the width direction faces upward in the unfolded state of the support portion, both sides of the front surface of the support portion in the width direction face upward . . . .

This transfer instrument can retract the support portion in the outer cylinder with the support portion deformed into a curved shape. It is therefore possible to reduce the diameter of the transfer instrument and achieve less invasion. Further, when the center portion of the front surface of the first support portion in the width direction faces upward in the unfolded state of the first support portion, both the sides of the front surface of the first support portion in the width direction face upward. With this configuration, the support portion has a simple shape without a fold line or the like, and is thus easily manufactured.

(2) The transfer instrument according to the above (1), in which the pair of separation portions may come into contact with each other on the back surface of the support portion with the support portion retracted in the outer cylinder.

The transfer tool can retract the support portion in the outer cylinder with the support portion deformed into a curved shape, so that it is possible to reduce the diameter of the transfer instrument and achieve less invasion. Further, the support portion has a simple shape without a fold line or the like, and is thus easily manufactured.

According to another aspect, a transfer instrument for transferring a medical sheet to a treatment site of a living body comprises: an outer cylinder having a distal-end opening at a distal end of the outer cylinder and a proximal-end opening at a proximal end of the outer cylinder, with the outer cylinder having an inner surface, and a carrier member that includes a shaft and a support portion, with the shaft having a distal end portion to which is fixed support portion so that the shaft and the support portion move together, and the support portion having a support surface and a back surface that is opposite the support surface. The shaft is positionable in the outer cylinder and is axially movable in a proximal direction relative to the outer cylinder to move the support portion from a first position in which the support portion is sheet-shaped to support the medical device on the support surface of the support portion and is located distal of the distal-end opening of the outer cylinder to a second position in which the support portion and the medical sheet are retracted into the outer cylinder. The shaft is also axially movable in a distal direction relative to the outer cylinder when the support portion is in the second position to move the support portion and the medical sheet from the second position to the first position so that the medical sheet supported on the support surface is removable from the support surface of the support portion. The support portion is configured so that when the support portion is in the second position retracted in the outer cylinder, the support portion is curved and includes, as viewed in a transverse cross-section, two spaced-apart convex shapes in which the back face of each convex shape faces the inner surface of the outer cylinder while the support surface of the support portion faces the medical sheet.

Another aspect involves a method comprising positioning a sheet-shaped support portion adjacent a medical sheet, wherein the sheet-shaped support portion has an upwardly facing support surface and a back surface opposite the support surface, with the sheet-shaped support portion being fixed to a distal end portion of a shaft, and the shaft passing through an outer cylinder that has an distal-end opening and a proximal-end opening so that a proximal portion of the shaft extends proximally beyond the proximal-end opening of the outer cylinder and the support portion is positioned distal of the distal-end opening of the outer cylinder. The method also comprises supporting the medical sheet on the support surface of the support portion; axially moving the shaft in a proximal direction relative to the outer cylinder to retract the support portion into the outer cylinder while the medical sheet is supported on the support portion; and the axially moving of the shaft in the proximal direction relative to the outer cylinder causing the support portion to deform into a curved shape inside the outer cylinder, with the curved shape of the support portion including a pair of separation portions spaced apart from an inner circumferential surface of the outer cylinder and protruding into the outer cylinder.

### BRIEF DESCRIPTION OF THE DRAWINGS

FIG. 1 is a perspective view of a transfer instrument according to an embodiment.

FIG. 2 is an exploded perspective view of the transfer instrument illustrated in FIG. 1.

FIG. 3 is a plan view of a distal end of the transfer instrument illustrated in FIG. 1.

FIG. 4 is a longitudinal cross-sectional view taken along line IV-IV in FIG. 3.

FIG. 5 is a transverse cross-sectional view taken along line V-V in FIG. 3.

FIG. 6 is a flowchart illustrating a procedure of a transfer method for transferring a medical sheet using the transfer instrument illustrated in FIG. 1.

FIG. 7 is an explanatory diagram of a sheet placing process.

FIG. 8 is an explanatory diagram of a retracting process.

FIG. 9 is a transverse cross-sectional view taken along line IX-IX in FIG. 8.

FIG. 10 is an explanatory diagram of a positioning process.

FIG. 11 is an explanatory diagram of an unfolding process.

FIG. 12 is an explanatory diagram of a moving process.

FIG. 13 is an explanatory diagram of a withdrawing process.

### DETAILED DESCRIPTION

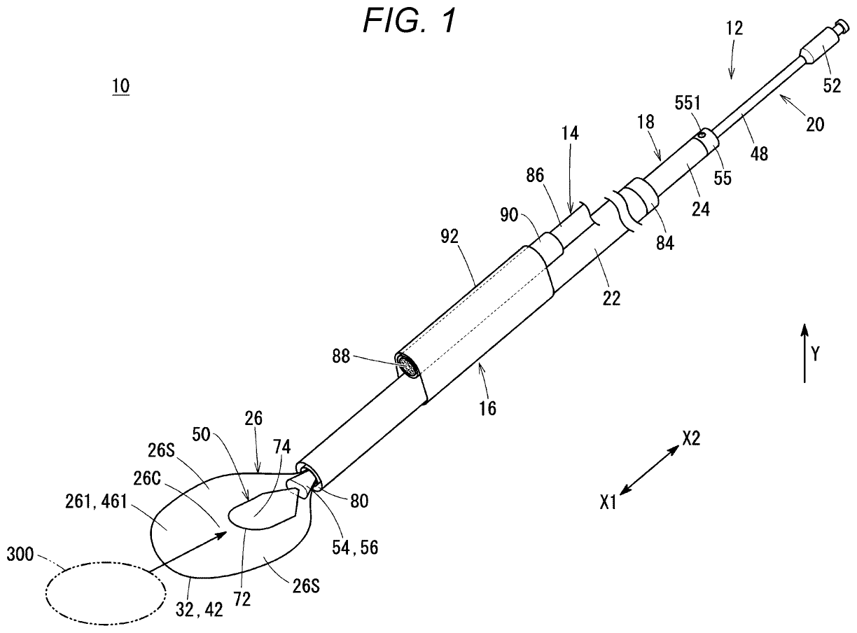

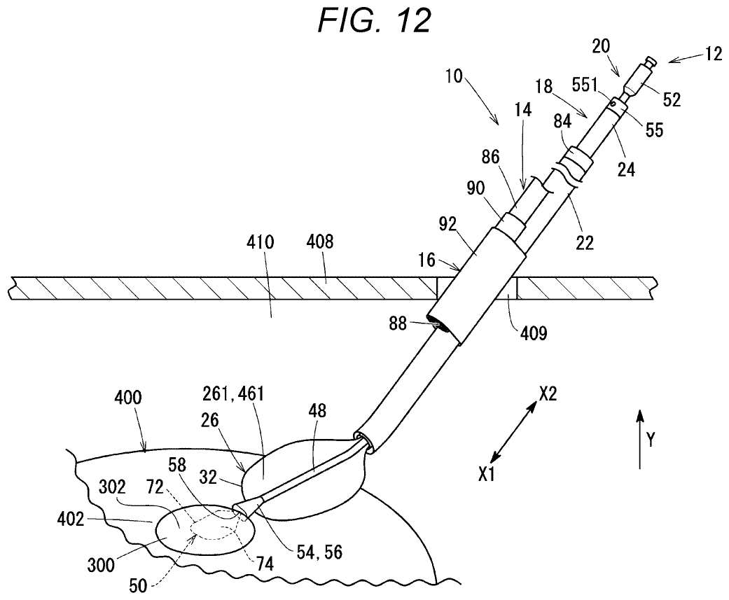

As illustrated in FIG. 1, a transfer instrument 10 according to the present embodiment is a medical instrument for transferring a medical sheet 300 to a treatment site of a living body. The transfer instrument 10 is used for, for example, the treatment of severe heart failure caused by ischemic heart disease. In this case, the medical sheet 300 is transplanted to a recipient site 402 of a heart 400 (the treatment site of the living body) (see FIG. 12). The transfer instrument 10 is capable of attaching a plurality of the medical sheets 300 to the recipient site 402.

Examples of such a medical sheet 300 include pharmaceutical products or regenerative medicine products for medical use, a medical instrument, and the like. The medical sheet 300 is formed in a sheet shape such as a film shape or a membrane shape (gel object). Fibrin or the like may be applied to the medical sheet 300 for reinforcement. Examples of the regenerative medicine products including cells include a cell sheet (sheet-shaped cell culture), a spheroid, and the like. It is possible to form the cell sheet by culturing autologous cells or allogenic cells. The cells constituting the cell sheet include, for example, somatic stem cells (adult stem cells), mesenchymal stem cells, or iPS cells (induced pluripotent stem cells)-derived cardiomyocytes. Examples of the somatic stem cells preferably include skeletal myoblast cells (myoblast cells).

The medical sheet 300 may contain a tissue adhesive, a local anesthetic, or the like. The medical sheet 300 has a thickness of, for example, about 100 μm, and has a diameter of, for example, about 60 mm. The thickness and the diameter (size) of the medical sheet 300 are not limited these values and can be set as desired.

The medical sheet 300 may be a sheet to be transplanted to an organ (for example, lung, liver, pancreas, kidney, small intestine, esophagus, or the like) other than the heart 400. Further, the medical sheet 300 may be, for example, an anti-adhesion sheet as long as the sheet is for medical use.

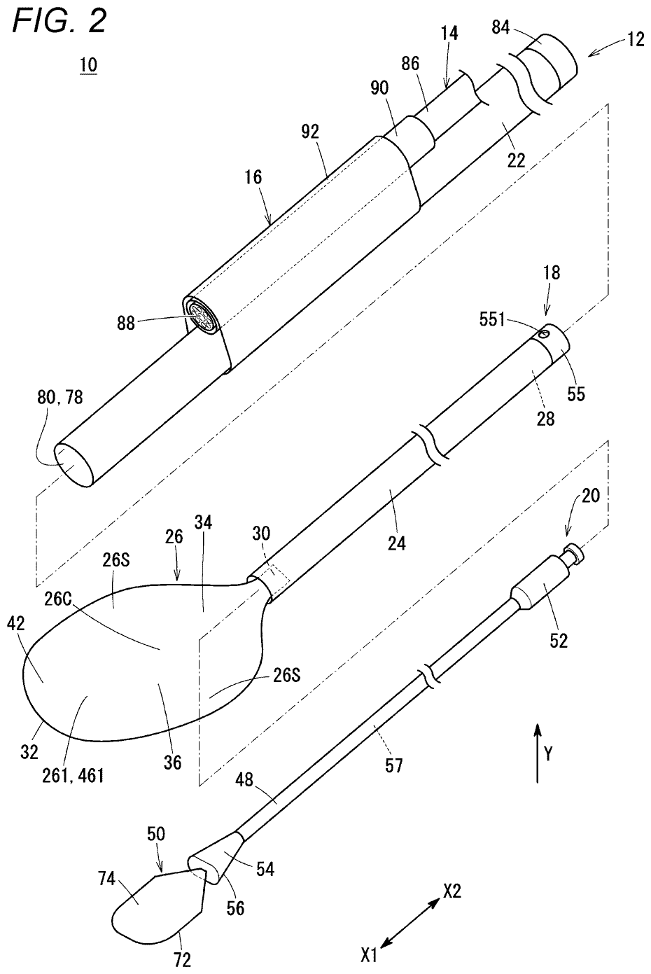

As illustrated in FIGS. 1 and 2, the transfer instrument 10 includes an instrument body 12, an endoscope 14, and a fixing member 16. The instrument body 12 includes a first carrier member 18, a second carrier member 20, and an outer cylinder 22. The transfer instrument 10 is not limited to a configuration including the endoscope 14.

In FIG. 2, the first carrier member 18 includes a first shaft 24 and a first support portion 26.

The first shaft 24 is a tubular body (in the present embodiment, a circular tube member) having a first lumen 28. The first lumen 28 opens at a distal end (end in a direction of arrow X1) of the first shaft 24 and opens at a proximal end (end in a direction of arrow X2) of the first shaft 24. The first shaft 24 has an airtight valve 55 provided at its proximal end with the valve 55 in close contact with an outer circumferential surface of the second shaft 48. The valve 55 has a marker 551 provided on its outer circumferential surface. When the transfer instrument 10 is used, the marker 551 is visible to the user. The first shaft 24 is not limited to a tubular body and may be a body other than a tubular body.

The first shaft 24 extends in an axial direction of the outer cylinder 22 and is disposed inside the outer cylinder 22 in a manner as to be movable along the axial direction. The first shaft 24 includes, for example, a resin material. Examples of the constituent material from which the first shaft 24 may be fabricated include, but not particularly limited to, polyethylene, polypropylene, fluororesin, polyethylene terephthalate, polymethyl methacrylate, a polyamide resin, polystyrene, polycarbonate, polyimide, polyetherimide, polyetheretherketone, polyvinyl chloride, an ABS resin, a polyamide elastomer, and a polyester elastomer. The first shaft 24 may include a metal material.

The first shaft 24 may be flexible. The first shaft 24 may have a flexible tube portion capable of maintaining a bent shape. In this case, the first shaft 24 can be bent into an any desired shape in a body cavity and can maintain the bent shape.

As illustrated in FIGS. 2 to 4, the first support portion 26 is attached to the distal end of the first shaft 24. The first support portion 26 includes or may be fabricated from, for example, a resin material. The first support portion 26 can hold the medical sheet 300. A flexible resin sheet member (film member) is bent into a predetermined form to form the first support portion 26. Alternatively, the sheet member is shaped into a predetermined form by a sheet forming die to form the first support portion 26. It is preferable that the sheet member have, but not particularly limited to, a thickness of, for example, 100 μm or more and 200 μm or less. The first support portion 26 includes a first joint 30 and a first support body 32.

The constituent material from which the first support portion 26 may be fabricated preferably has transparency, and examples of the constituent material include, but are not particularly limited to, polyethylene, polycarbonate, polyamide, polystyrene, polypropylene, polyacetal resin, polyimide, polyetherimide, polyetheretherketone, polyethylene terephthalate, and fluororesin. Further, the first support portion 26 may have a mesh shape.

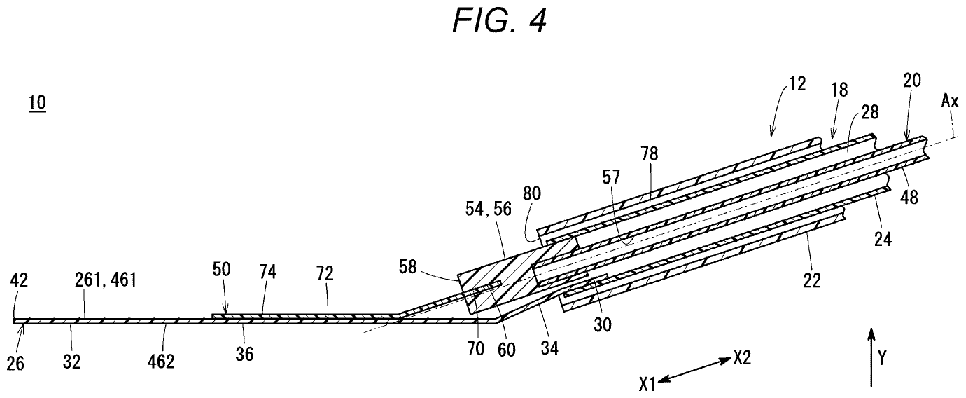

In FIG. 4, the first joint 30 is bonded to an inner circumferential surface of the distal end of the first shaft 24 with an adhesive. Examples of the adhesive include, but are not particularly limited to, a UV adhesive, a hot-melt adhesive, and an instant adhesive (for example, cyanoacrylate-based instant adhesive). The first joint 30 may be thermally fused to the inner circumferential surface of the first shaft 24. The first support portion 26 may be detachable from the distal end of the first shaft 24.

As illustrated in FIG. 2, the first support body 32 extends in a distal end direction from first joint 30. The first support body 32 includes a proximal-end support portion 34, an intermediate support portion 36, and a distal-end support portion 42. The first support body 32 includes a first support surface 261. The marker 551 of the first shaft 24 is disposed so as to face upward when the first support surface 261 faces upward. That is, the marker 551 allows the user to confirm the direction of the first support surface 261 of the first support portion 26.

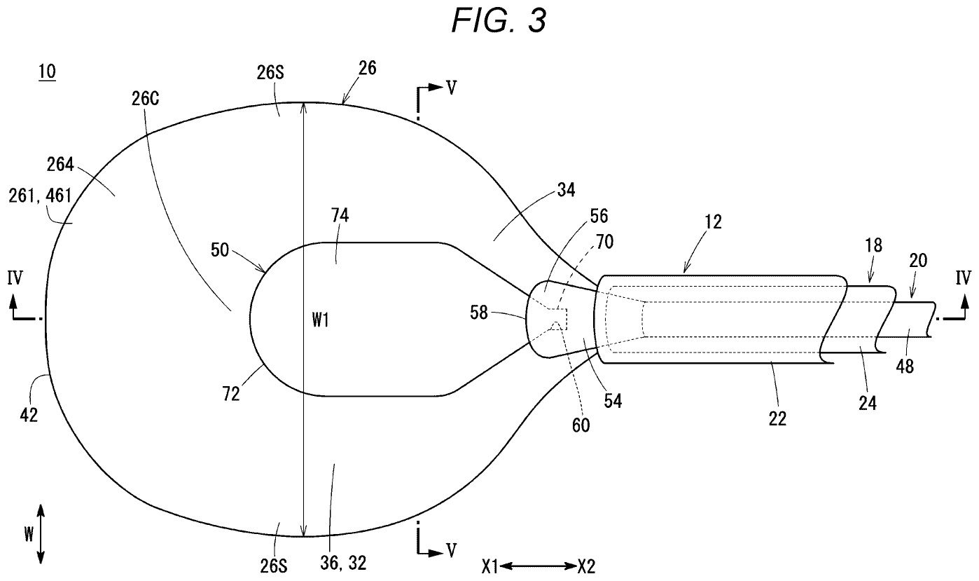

As illustrated in FIG. 3, the proximal-end support portion 34 is formed to be wider in its extending direction. In other words, both sides of the proximal-end support portion 34 in a width direction are tapered toward the first joint 30 (see FIG. 2). The intermediate support portion 36 is formed in a tapered shape so as to be gradually narrower in width from the distal end toward the proximal end (in the direction of arrow X2).

The distal-end support portion 42 is connected to the distal end of the intermediate support portion 36. The distal-end support portion 42 protrudes in an arc shape in the distal end direction (the direction of arrow X1). That is, the distal-end support portion 42 of the first support portion 26 has an arc shape when viewed from a direction orthogonal to the first support surface 261 illustrated in FIG. 3.

As illustrated in FIG. 4, the proximal-end support portion 34 extends roughly along an axis Ax of the first shaft 24 from a distal end of the first joint 30 in the distal end direction (the direction of arrow X1). The intermediate support portion 36 intersects with the axis Ax of the first shaft 24 and extends from a distal end of the proximal-end support portion 34 toward the distal end of the first support portion 26 (in the direction of arrow X1).

The first support body 32 includes a front surface 461 that faces upward (in the direction of arrow Y) and includes the first support surface 261, and a back surface 462 that is a surface opposite to the front surface 461. The first support surface 261 includes a flat surface continuous over an upper surface of the proximal-end support portion 34 and upper surfaces of the intermediate support portion 36 and the distal-end support portion 42. A lubricant may be applied to the first support surface 261 so as to allow a second support portion 50 (to be described later) of the second carrier member 20 to smoothly slide on the first support surface 261.

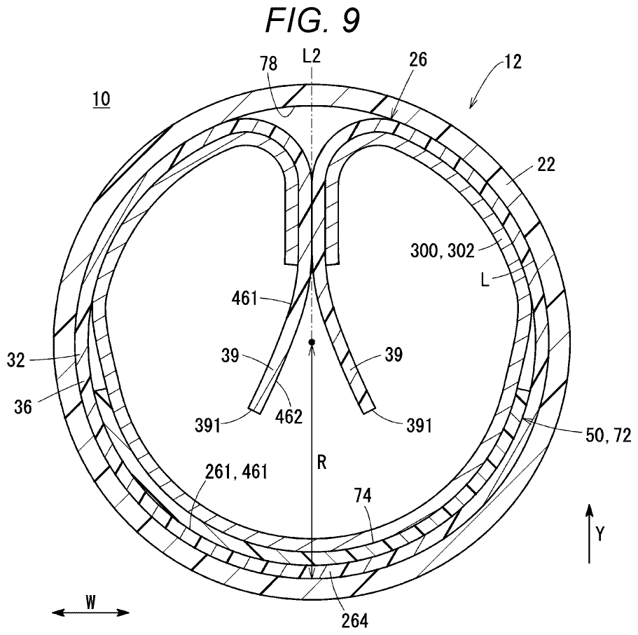

In FIG. 3, the direction (W direction) orthogonal to the axial direction of the first shaft 24 refers to the width direction of the first support portion 26. The W direction also refers to a width direction of other components (the outer cylinder 22 and the like). W1 in FIG. 3 denotes a width of the widest portion of the first support portion 26. The width W1 of the first support portion 26 is larger than an inner circumferential length L of the outer cylinder 22 (see FIG. 9). In FIG. 9, the inner circumferential length L of the outer cylinder 22 is a length of an inner circumferential surface along a circumferential direction of the outer cylinder 22 (L=2πR). R denotes the radius of the lumen 78 of the outer cylinder 22.

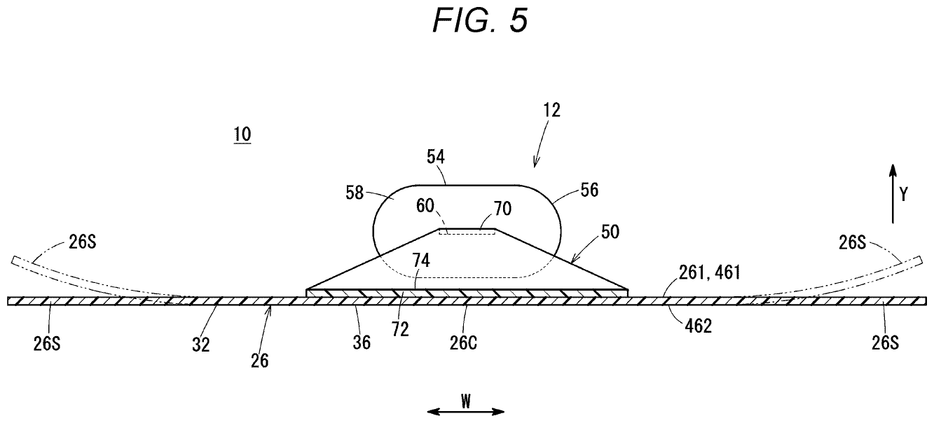

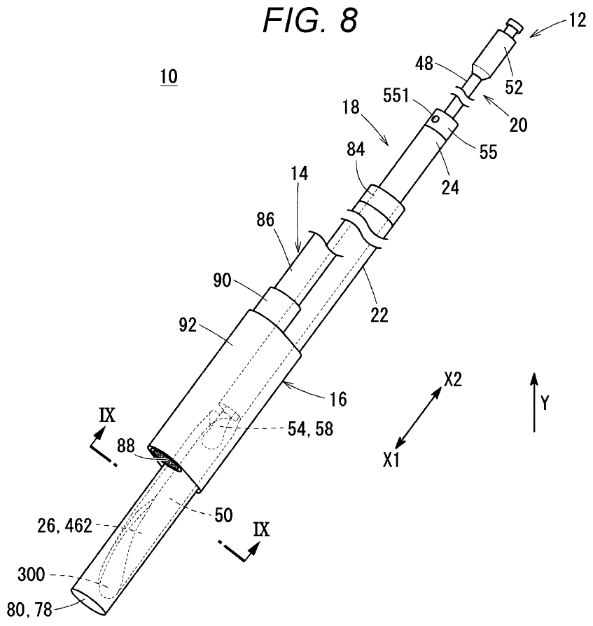

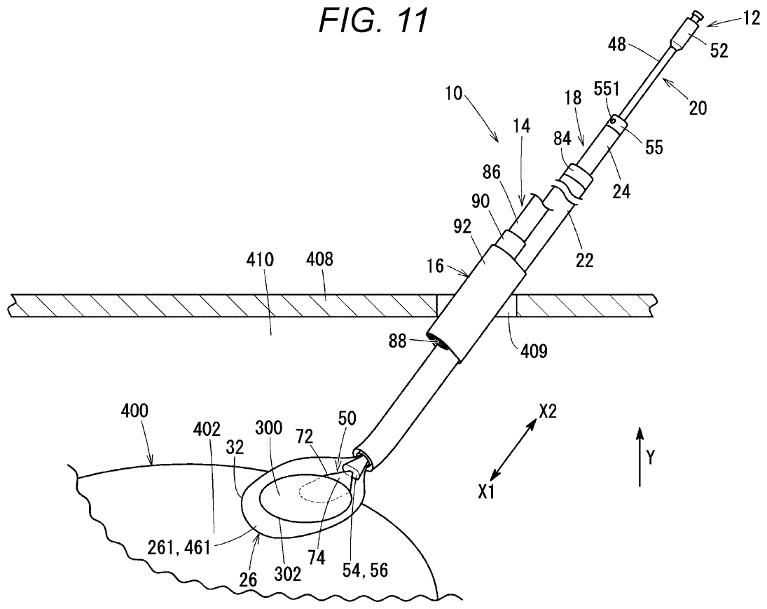

As illustrated in FIG. 1, the first support portion 26 has a roughly flat plate shape with the first support portion 26 unfolded by protruding from a distal-end opening 80 of the outer cylinder 22 (hereinafter, also referred to as “unfolded state of the first support portion 26”). Therefore, as illustrated in FIG. 5, when a center portion 26C of the front surface 461 of the first support portion 26 in the width direction faces upward in the unfolded state of the first support portion 26, both sides 26S of the front surface 461 of the first support portion 26 in the width direction face upward. After the first support portion 26 is retracted in the outer cylinder 22 as illustrated in FIG. 8, when the first support portion 26 protrudes from the distal-end opening 80 of the outer cylinder 22 to return to the unfolded state as illustrated in FIG. 11, the first support portion 26 may remain somewhat curved. No fold line, however, is formed on the first support portion 26. Therefore, as indicated by an imaginary line in FIG. 5, in the unfolded state of the first support portion 26, the first support portion 26 may be somewhat curved so as to make both the sides 26S of the first support portion 26 in the width direction slightly higher than the center portion 26C. Even in this case, both the sides 26S of the front surface 461 of the first support portion 26 in the width direction face upward.

As illustrated in FIG. 2, the second carrier member 20 includes a second shaft 48, the second support portion 50, and a hub 52.

The second shaft 48 is a tubular body (in the present embodiment, a circular tube member) having a second lumen 57. The second shaft 48 is longer in the axial direction than the first shaft 24. The second shaft 48 is inserted into the first lumen 28 of the first shaft 24 (see FIGS. 1 and 4). In other words, a distal end of the second shaft 48 protrudes in the distal end direction (the direction of arrow X1) relative to the distal-end opening of the first shaft 24. A proximal end of the second shaft 48 protrudes in the proximal end direction (the direction of arrow X2) relative to the proximal-end opening of the first shaft 24 (see FIG. 1). The second shaft 48 is provided extending along the first shaft 24 so as to be movable along the first shaft 24. Note that the second shaft 48 is not limited to a tubular body and may be a body other than a tubular body.

The second shaft 48 is configured to follow the shape of the first support portion 26. As the constituent material of the second shaft 48, for example, a material more flexible than the constituent material of the first shaft 24 is selected. Specifically, examples of the constituent material of the second shaft 48 include a polyamide elastomer, a polyester elastomer, a polyurethane elastomer, polyvinyl chloride, polybutadiene, a silicone rubber, and a metal coil (including a composite with a resin). The second shaft 48 is flexible.

As illustrated in FIG. 4, the second shaft 48 includes a carrier holding portion 54 and a pressure-application portion 56 that is the distal end of the second shaft 48. The pressure-application portion 56 includes an elastic body such as an elastomer member. The pressure-application portion 56 presses the first support portion 26 against an inner surface of the outer cylinder 22 with the first support portion 26 retracted in the outer cylinder 22.

A distal end of the carrier holding portion 54 includes a pressing surface 58. The carrier holding portion 54 can cause the pressing surface 58 to press an outer edge surface of the medical sheet 300 supported by the first support portion 26 in the distal end direction (the direction of arrow X1). In the present embodiment, the pressure-application portion 56 is provided with the carrier holding portion 54 that supports the second support portion 50. The carrier holding portion 54 includes the pressing surface 58 and an attachment hole 60.

In FIG. 4, the pressing surface 58 is provided on a distal end surface of the carrier holding portion 54. The attachment hole 60 opens at the pressing surface 58. The second support portion 50 is attached to the pressing surface 58. The pressing surface 58 presses the outer edge surface of the medical sheet 300 in the distal end direction (the direction of arrow X1) (see FIG. 12).

In FIGS. 2 to 4, the second support portion 50 is a flexible sheet. The second support portion 50 includes a second joint 70 and a second support body 72. The second joint 70 is provided at a proximal end of the second support portion 50. The second joint 70 is provided at a proximal end of the second support body 72. The second joint 70 is inserted into or positioned in the attachment hole 60 of the carrier holding portion 54, and is bonded, for example. The second joint 70 may be joined to the attachment hole 60 of the carrier holding portion 54 by a suitable joining method other than adhesion. The second support portion 50 may be integrally formed with the carrier holding portion 54.

The second support body 72 extends in the distal end direction (the direction of arrow X1) from the second joint 70. The second support body 72 extending from the second joint 70 is shorter in the extending direction than the first support body 32 extending from the first joint 30. The second support body 72 is provided, on its upper surface, with a second support surface 74 on which the medical sheet 300 is placed. The second support surface 74 includes a flat surface. The second support body 72 is smaller than the first support body 32. That is, the second support surface 74 is smaller in area than the first support surface 261.

In FIG. 2, the hub 52 is attached to the proximal end of the second shaft 48.

In FIGS. 1 and 2, the outer cylinder 22 is a cylindrical member having a lumen 78. The lumen 78 has a distal-end opening 80 that opens at a distal end (end in the direction of arrow X1) of the outer cylinder 22. The lumen 78 opens at a proximal end (end in the direction of arrow X2) of the outer cylinder 22. The outer cylinder 22 is flexible. Examples of the constituent material from which the outer cylinder 22 may be fabricated are the same as the examples of the constituent material of the first shaft 24 described above. The first shaft 24 is inserted into the lumen 78 of the outer cylinder 22. The outer cylinder 22 is shorter in length along the axial direction than the first shaft 24. The outer cylinder 22 is provided with, at its proximal end, an airtight valve 84 that is in close contact with an outer circumferential surface of the first shaft 24.

In FIGS. 2 and 4, a distal end surface of the outer cylinder 22 extends orthogonal to the axial direction of the outer cylinder 22.

As illustrated in FIG. 2, the endoscope 14 includes a long or elongated endoscope main body 86. A distal end of the endoscope main body 86 is fixed to an outer circumferential surface of the outer cylinder 22 by the fixing member 16 (see FIG. 1). An objective lens 88 provided on a distal end surface of the endoscope main body 86 is oriented toward the distal end of the outer cylinder 22 (in the direction of arrow X1). The distal end of the endoscope main body 86 is fixed to an intermediate portion of the outer cylinder 22 in the axial direction. The distal end of the endoscope main body 86 may be fixed to the distal end of the outer cylinder 22.

The fixing member 16 includes, for example, a fixing cylinder 90 and a fixing tube 92. The fixing cylinder 90 includes, for example, a hard resin material. The endoscope main body 86 can be inserted into or positioned in a lumen of the fixing cylinder 90. The fixing cylinder 90 is disposed along a longitudinal direction of the outer cylinder 22. The fixing tube 92 is a tube for fixing the fixing cylinder 90 at a predetermined position of the outer cylinder 22. The fixing tube 92 is, for example, a heat-shrink tube. The manner in which the distal end of the endoscope main body 86 is fixed to the outer cylinder 22 may be determined as desired.

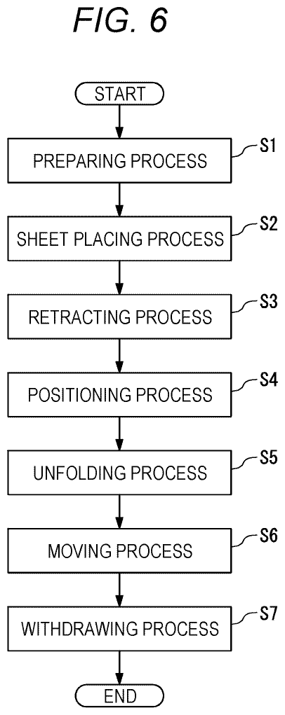

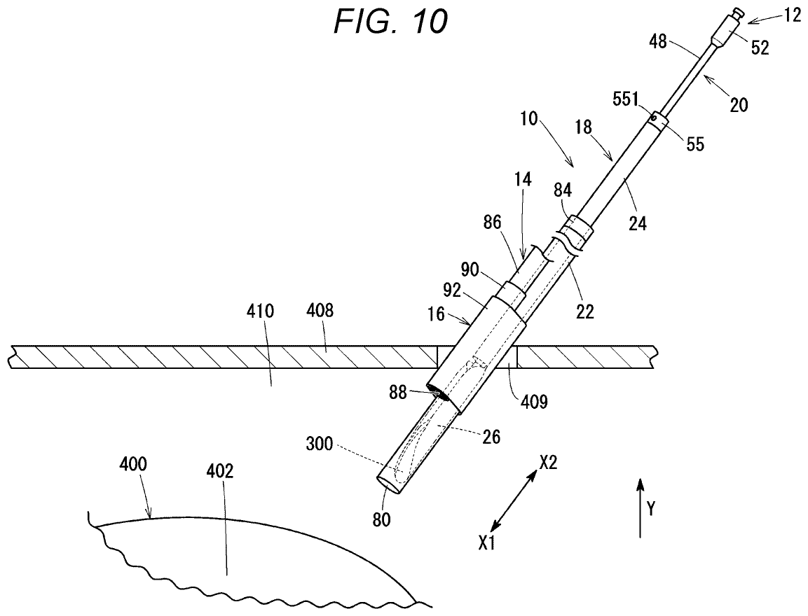

Next, a transfer method for transferring the medical sheet 300 to a treatment site of a living body will be described. Specifically, as illustrated in FIGS. 10 to 13, the transfer method for transferring the medical sheet 300 to the recipient site 402 of the heart 400 (the treatment site of the living body) during thoracoscopic surgery will be described. As illustrated in FIG. 6, the transfer method according to the present embodiment includes a preparing process, a sheet placing process, a retracting process, a positioning process, an unfolding process, a moving process, and a withdrawing process.

First, in the preparing process (step S1), the transfer instrument 10 according to the present embodiment described above is prepared. The following description will be given on the assumption that a state as illustrated in FIG. 1 is an initial state of the transfer instrument 10. In the initial state, the first shaft 24 and the second shaft 48 are moved in the distal end direction (the direction of arrow X1) relative to the outer cylinder 22 so as to be at a protruding position (second position) where the first support portion 26 and the second support portion 50 protrude from the distal-end opening 80 of the outer cylinder 22 in the distal end direction. The first and second support portions 26 and 50 are each unfolded when moving out of the outer cylinder 22 in the distal end direction, and the second support portion 50 is positioned on the first support surface 261 of the first support portion 26. That is, the second support portion 50 is positioned at a retracted position where the second support portion 50 is placed on the first support surface 261 of the first support portion 26. At this time, a proximal end of the carrier holding portion 54 is in the first lumen 28 of the first shaft 24.

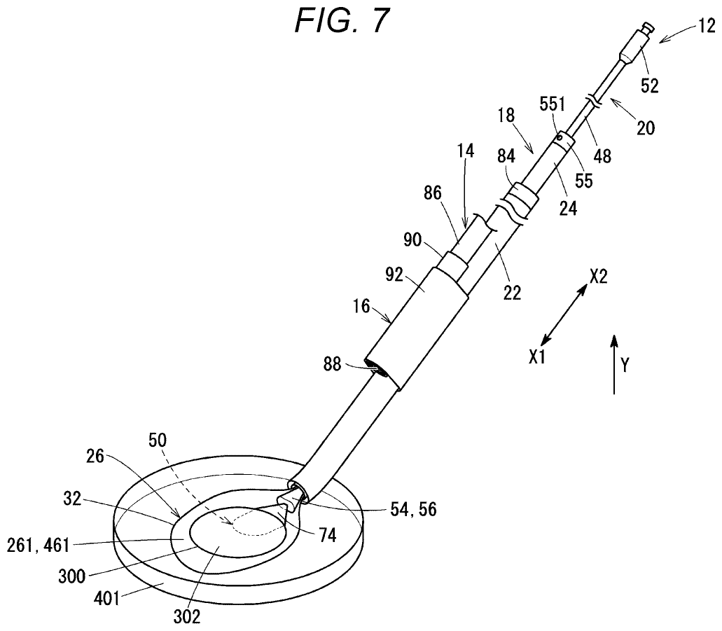

Next, in the sheet placing process (step S2), as illustrated in FIG. 7, the medical sheet 300 placed on or located in a Petri dish 401 is placed on the second support surface 74. The medical sheet 300 protrudes outward from the second support portion 50 with the medical sheet 300 placed on the second support surface 74. The first support surface 261 supports an overhanging portion 302 of the medical sheet 300 that sticks out over the second support portion 50.

Subsequently, in the retracting process (step S3 in FIG. 6), as illustrated in FIG. 8, the medical sheet 300 is retracted in the outer cylinder 22 together with the first support portion 26 and the second support portion 50 at a retracted position (first position).

Specifically, in the retracting process, the user deforms the first support portion 26 into a curved shape illustrated in FIG. 9 using, for example, an appropriate instrument or his/her hand, and moves the first shaft 24 of the first carrier member 18 and the second shaft 48 of the second carrier member 20 together in the proximal end direction (the direction of arrow X2) relative to the outer cylinder 22 while maintaining the curved shape. Accordingly, as illustrated in FIG. 9, the first support portion 26 is retracted in the outer cylinder 22 with the first support portion 26 deformed into a curved shape.

With the first support portion 26 retracted in the outer cylinder 22, the intermediate support portion 36 is deformed into a cylindrical shape along the inner surface of the outer cylinder 22. Both ends of the first support portion 26 in the width direction are curved so as to cause the front surface 461 of the first support portion 26 to face inward and the back surface 462 of the first support portion 26 to face outward. The back surfaces 462 on both sides of the first support portion 26 in the width direction face each other across an imaginary line L2 extending in a direction orthogonal to the center axis of the outer cylinder 22. Both the ends of the first support portion 26 in the width direction protrude downward (toward the first support surface 261 and the front surface 461).

Accordingly, the back surface 462 of the first support portion 26 is curved in close contact with the inner surface of the outer cylinder 22, both the ends of the first support portion 26 are further curved to fold back toward the center of the outer cylinder 22, and free ends of both the ends are positioned below the center axis of the outer cylinder 22. That is, the first support portion 26 is retracted in the outer cylinder 22 with the first support portion 26 curved in a heart shape along the inner surface of the outer cylinder 22.

As illustrated in FIG. 9, with the first support portion 26 retracted in the outer cylinder 22 (hereinafter, also referred to as “curved state of the first support portion 26”), the first support portion 26 has a pair of separation portions 39 that are spaced apart from the inner circumferential surface of the outer cylinder 22 and protrude into the outer cylinder 22 (lumen 78). The pair of separation portions 39 are in contact with each other on the back surface 462 of the first support portion 26. That is, the back surface 462 of the separation portions 39 of the first support portion 26 are in contact with each other. In the aspect illustrated in FIG. 9, in the curved state of the first support portion 26, a separation distance between lower portions of the pair of separation portions 39 increases downward. Therefore, free ends 391 of the pair of separation portions 39 are spaced apart from each other. In the curved state of the first support portion 26, the lower portions of the pair of separation portions 39 may be parallel to each other along the imaginary line L2.

The heart shape refers to a roughly round shape including two convex shapes, a convex shape on one side and a convex shape on the other side. In a case where the heart shape is formed in the lumen 78 of the tubular body (outer cylinder 22), the two convex shapes protruding toward the opposite sides along the inner surface of the tubular body come close to each other, so that the entire contour becomes a roughly round shape along the inner surface of the tubular body (refer to the shape of the first support portion 26 in FIG. 9).

Along with the curved deformation of the first support portion 26, the second support portion 50 (see FIGS. 1 to 3) is similarly curved and deformed along the first support portion 26 inside the first support portion 26 (on the front surface 461 side). Along with the curved deformation of the first support portion 26 and the second support portion 50, the medical sheet 300 is deformed into a shape corresponding to the shape of the first support body 32 and the shape of the second support body 72, and the medical sheet 300 is retracted in the outer cylinder 22 accordingly.

As illustrated in FIG. 8, when the first support portion 26 is entirely inserted into or positioned in the outer cylinder 22, the retracting process is complete. At the completion of the retracting process, the first support portion 26 may partially protrude from the distal-end opening 80 of the outer cylinder 22. In this case, the state where the first support portion 26 partially protrudes from the distal-end opening 80 of the outer cylinder 22 corresponds to the first position of the first support portion 26.

Subsequently, in the positioning process (step S4 in FIG. 6), as illustrated in FIG. 10, the transfer instrument 10 is inserted into a chest cavity 410 through an incision 409 in a chest 408. At this time, the distal end of the transfer instrument 10 is positioned near the recipient site 402 of the heart 400, and the distal end of the endoscope 14 is positioned in the chest cavity 410. Before the transfer instrument 10 is inserted into the chest cavity 410, a liquid supply instrument (not illustrated) may be connected to a connection port of the hub 52 to introduce a liquid (for example, a physiological saline solution).

Subsequently, in the unfolding process (step S5 in FIG. 6), as illustrated in FIG. 11, the first support portion 26, the second support portion 50, and the medical sheet 300 are unfolded. Specifically, in the unfolding process, with the first shaft 24 gripped, the first shaft 24 is moved in the distal end direction (the direction of arrow X1) relative to the outer cylinder 22. Accordingly, the valve 55 of the first shaft 24 causes the second shaft 48 to move together with the first shaft 24 in the distal end direction (the direction of arrow X1). Then, the first support portion 26 that has moved out of the distal-end opening 80 of the outer cylinder 22 returns to the original shape due to its restoring force. At the second position where the first support portion 26 is unfolded, the first support portion 26 and the second support portion 50 unroll flat.

In the unfolding process, of the second carrier member 20, the second support surface 74 on which the medical sheet 300 is placed is entirely positioned on the first support surface 261. At this time, the medical sheet 300 is supported by the first support surface 261 and the second support surface 74. It is therefore possible to prevent the overhanging portion 302 of the medical sheet 300 from becoming crinkled before the medical sheet 300 is transferred to the recipient site 402 of the heart 400.

Next, in the moving process (step S6 in FIG. 6), as illustrated in FIG. 12, the second carrier member 20 is moved in the distal end direction (the direction of arrow X1) relative to the first carrier member 18, so that the second support portion 50 on which the medical sheet 300 is placed is moved from the retracted position to an advanced position, and the second support portion 50 protrudes in the distal end direction (the direction of arrow X1) relative to the distal end of the first support portion 26. Specifically, in the moving process, the second shaft 48 is moved in the distal end direction relative to the first shaft 24.

Accordingly, the second support portion 50 moves in the distal end direction (the direction of arrow X1) relative to the first support portion 26. At this time, when the distal end surface of the carrier holding portion 54 (pressure-application portion 56) presses the outer edge surface of the medical sheet 300 in the distal end direction, the medical sheet 300 is entirely positioned further than the first support portion 26 in the distal end direction. In this moving process, the medical sheet 300 is moved to above the recipient site 402 of the heart 400 to bring the overhanging portion 302 of the medical sheet 300 into contact with the recipient site 402.

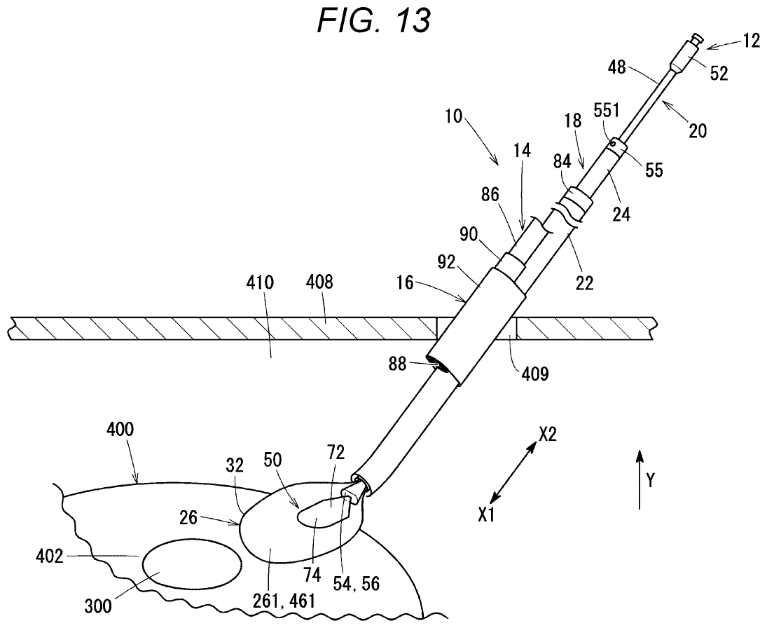

Subsequently, in the withdrawing process (step S7 in FIG. 6), as illustrated in FIG. 13, the second carrier member 20 is moved from the second position to the first position to withdraw the second support portion 50 from between the recipient site 402 and the medical sheet 300. Accordingly, the medical sheet 300 entirely comes into contact with the surface of the recipient site 402. This is the end of the transfer of the medical sheet 300 to the recipient site 402. Subsequently, the transfer instrument 10 is withdrawn from the chest 408 with the first support portion 26 and the second support portion 50 retracted in the outer cylinder 22.

The present embodiment has the following effects.

As illustrated in FIG. 9, the transfer instrument 10 can cause the first support portion 26 to retract in the outer cylinder 22 with the first support portion deformed into a curved shape. It is therefore possible to reduce the diameter of the transfer instrument 10 and achieve less invasion.

As illustrated in FIG. 5, when the center portion 26C of the front surface 461 of the first support portion 26 in the width direction faces upward in the unfolded state of the first support portion 26, both the sides 26S of the front surface 461 of the first support portion 26 in the width direction face upward. That is, the first support portion 26 has a roughly flat shape. With this configuration, the first support portion 26 has a simple shape without a fold line or the like, and is thus easily manufactured.

The present invention is not limited to the above-described disclosure, and various configurations can be adopted without departing from the gist of the present invention. For example, the second support portion 50 may be removed from the second carrier member 20. The second carrier member 20 may be removed from the transfer instrument 10, and the medical sheet 300 may be moved from above the first support portion 26 onto the recipient site 402 using a different instrument.

The detailed description above describes embodiments of a transfer instrument and transfer method for transferring a medical sheet to a treatment site of a living body. The described embodiments represent examples of the new transfer instrument and transfer method disclosed here, but the invention is not limited to the precise embodiments and variations described. Various changes, modifications and equivalents can be effected by one skilled in the art without departing from the spirit and scope of the invention as defined in the accompanying claims. It is expressly intended that all such changes, modifications and equivalents that fall within the scope of the claims are embraced by the claims.

What is claimed is:

1. A transfer instrument used to transfer a medical sheet to a treatment site of a living body, the transfer instrument comprising:
an outer cylinder including a distal-end opening;
a carrier member including a shaft and a support portion, the shaft being positionable in the outer cylinder so that the shaft extends in an axial direction of the outer cylinder and is movable along the axial direction relative to the outer cylinder, the support portion being sheet-shaped and being disposed at a distal end of the shaft;
the support portion including a support surface configured to hold the medical sheet, a front surface including the support surface, and a back surface opposite to the front surface, the support portion having a width in a width direction orthogonal to an extending direction of the shaft, the width of the support portion being larger than an inner circumferential length of the outer cylinder;
the support portion being positionable in a first position retracted in the outer cylinder with the support portion deformed into a curved shape, the curved shape of the support portion in the first position including a pair of separation portions spaced apart from an inner circumferential surface of the outer cylinder and protruding into the outer cylinder;
the support portion being movable in a distal direction relative to the outer cylinder from the first position to a second position, in which second position the support portion protrudes distally beyond the distal-end opening of the outer cylinder and is unfolded in an unfolded state; and
when a center portion of the front surface of the support portion in the width direction faces upward in the unfolded state of the support portion, both sides of the front surface of the support portion in the width direction face upward.

2. The transfer instrument according to claim 1, wherein the pair of separation portions are in contact with each other on the back surface of the support portion when the support portion is in the first position in which the support portion is retracted in the outer cylinder.

3. The transfer instrument according to claim 1, wherein the separation portions each include a free end portion terminating at a free end, the free end portions of the pair of separation portions being spaced from one another and diverging away from one another.

4. The transfer instrument according to claim 1, wherein the outer cylinder possesses an inner radius R and an inner circumferential length L, wherein L=2πR, the support portion having a widest portion in which the width of the support portion is widest, the width of the widest portion of the support portion is larger than the inner circumferential length L of the outer cylinder.

5. The transfer instrument according to claim 1, wherein the shaft includes a proximal end portion that extends proximally beyond a proximal open end of the outer cylinder when the support position is in the first position and when the support position is in the second position.

6. The transfer instrument according to claim 1, wherein an outer circumferential surface of a proximal portion of the shaft includes a marker that faces upward when the front surface of the second portion faces upward to allows a user of the transfer instrument to confirm a direction of orientation of the front surface of the support portion.

7. A transfer instrument for transferring a medical sheet to a treatment site of a living body, the transfer instrument comprising:
an outer cylinder having a distal-end opening at a distal end of the outer cylinder and a proximal-end opening at a proximal end of the outer cylinder, the outer cylinder having an inner surface;
a carrier member that includes a shaft and a support portion, the shaft having a distal end portion to which is fixed support portion so that the shaft and the support portion move together, the support portion having a support surface and a back surface that is opposite the support surface;
the shaft being positionable in the outer cylinder and being axially movable in a proximal direction relative to the outer cylinder to move the support portion from a first position in which the support portion is sheet-shaped to support the medical device on the support surface of the support portion and is located distal of the distal-end opening of the outer cylinder to a second position in which the support portion and the medical sheet are retracted into the outer cylinder;
the shaft being axially movable in a distal direction relative to the outer cylinder when the support portion is in the second position to move the support portion and the medical sheet from the second position to the first position so that the medical sheet supported on the support surface is removable from the support surface of the support portion; and
the support portion being configured so that when the support portion is in the second position retracted in the outer cylinder, the support portion is curved and includes, as viewed in a transverse cross-section, two spaced-apart convex shapes in which the back face of each convex shape faces the inner surface of the outer cylinder while the support surface of the support portion faces the medical sheet.

8. The transfer instrument according to claim 7, wherein the outer cylinder possesses an inner radius R and an inner circumferential length L, wherein L=2πR, the support portion having a widest portion in which the width of the support portion is widest, the width of the widest portion of the support portion is larger than the inner circumferential length L of the outer cylinder.

9. The transfer instrument according to claim 7, wherein the shaft includes a proximal end portion that extends proximally beyond a proximal open end of the outer cylinder when the support position is in the first position and when the support position is in the second position.

10. The transfer instrument according to claim 7, wherein an outer circumferential surface of a proximal portion of the shaft includes a marker that faces upward when the front surface of the second portion faces upward to allows a user of the transfer instrument to confirm a direction of orientation of the front surface of the support portion.

11. The transfer instrument according to claim 7, wherein the support portion that is curved includes, as viewed in the transverse cross-section, two separation portions positioned between the two spaced-apart convex shapes.

12. The transfer instrument according to claim 11, wherein the two separation portions extend away from the inner surface of the outer cylinder toward a center of an interior of the outer cylinder.

13. The transfer instrument according to claim 11, wherein the two separation portions of the support portion include one part in which the back surfaces of the two separation portions are in contact with one another and another part in which the back surfaces of the two separation portions are spaced apart from one another.

14. The transfer instrument according to claim 11, wherein each of the two separation portions extends away from the inner surface of the outer cylinder, includes a free end portion and terminates at free end of the separation portion, the free end portions of the two separation portions being spaced from one another and diverging away from one another.

15. A method comprising:
positioning a sheet-shaped support portion adjacent a medical sheet, the sheet-shaped support portion having an upwardly facing support surface and a back surface opposite the support surface, the sheet-shaped support portion being fixed to a distal end portion of a shaft, the shaft passing through an outer cylinder that has an distal-end opening and a proximal-end opening so that a proximal portion of the shaft extends proximally beyond the proximal-end opening of the outer cylinder and the support portion is positioned distal of the distal-end opening of the outer cylinder;
supporting the medical sheet on the support surface of the support portion;
axially moving the shaft in a proximal direction relative to the outer cylinder to retract the support portion into the outer cylinder while the medical sheet is supported on the support portion; and
the axially moving of the shaft in the proximal direction relative to the outer cylinder causing the support portion to deform into a curved shape inside the outer cylinder, the curved shape of the support portion including a pair of separation portions spaced apart from an inner circumferential surface of the outer cylinder and protruding into the outer cylinder.

16. The method according to claim 15, further comprising axially moving the shaft in a distal direction relative to the outer cylinder to advance the support portion and the medical sheet from inside the outer cylinder to an extended position in which the support portion is positioned distal of the distal-end opening of the outer cylinder, the support portion changing from the curved shape toward the sheet-shape when the support portion and the medical sheet are advanced from inside the outer cylinder to the extended position.

17. The method according to claim 15, wherein the curved shape of the support portion located inside the outer cylinder includes, as viewed in a transverse cross-section, two spaced-apart convex shapes in which the back surface of each convex shape faces the inner circumferential surface of the outer cylinder while the support surface of the support portion faces the medical sheet.

18. The method according to claim 15, wherein the pair of separation portions are in contact with each other on the back surface of the support portion when the support portion is in the outer cylinder.

19. The method according to claim 15, wherein the separation portions each include a free end portion terminating at a free end, the free end portions of the pair of separation portions being spaced from one another and diverging away from one another.
